(19) United States
(12) Patent Application Publication
(10) Pub. No.: US 2018/0111057 A1
(43) Pub. Date: Apr. 26, 2018

(54) METHOD OF EXTRACTION OF BERRIES OR FRUITS USING AN ALKALINE MAGNESIUM COMPOUND FOR PHARMACEUTICAL, HERBAL, NUTRITIONAL OR COSMETIC INGREDIENTS

(71) Applicant: Mika Tapio REIJONEN, Helsinki (FI)

(72) Inventor: Mika Tapio REIJONEN, Helsinki (FI)

(21) Appl. No.: 15/558,960

(22) PCT Filed: Mar. 15, 2016
(86) PCT No.: PCT/FI2016/000007

§ 371 Date: Sep. 15, 2017

(30) Foreign Application Priority Data
Mar. 16, 2015 (FI) FI20150078

(51) Int. Cl. B01D 11/02; A23L 19/00; A23L 33/105; A23L 2/04; A23L 2/52; A61K 36/87; A61K 36/185

(52) U.S. Cl. CPC B01D 11/0288; A23L 19/01; A23L 33/105; A23L 2/04; A61K 2236/17; A61K 36/87; A61K 36/185; A23V 2002/00; A61K 2236/37; A23L 2/52

(57) ABSTRACT

The invention is a method of extraction of berries or fruits of (1 a)) with a solvent (1 c)), typically water, and also an alkaline magnesium compound (1 b)) and heating a mixture (1 d)), the mixture is allowed to cool and is obtained berry and fruit materials in soluble form (1 e)) which is decanted or used wholly in the pharmaceutical, dietary supplement, herbal, or cosmetic product ingredients.

### CROSS-REFERENCE

The present utility application is the National Phase filing under 35 U.S.C. 371 of International Application No. PCT/FI2016/000007, entitled “METHOD OF EXTRACTION OF BERRIES OR FRUITS USING AN ALKALINE MAGNESIUM COMPOUND FOR PHARMACEUTICAL, HERBAL, NUTRITIONAL OR COSMETIC INGREDIENTS”, filed 15 Mar. 2016 and foreign patent application serial number FI20150078 filed on 15 Mar. 2015 entitled “METHOD OF EXTRACTION OF BERRIES OR FRUITS USING AN ALKALINE MAGNESIUM COMPOUND FOR PHARMACEUTICAL, HERBAL, NUTRITIONAL OR COSMETIC INGREDIENTS under 35 U.S.C. 119(a). The contents of these related applications are incorporated herein by reference for all purposes to the extent that such subject matter is not inconsistent herewith or limiting hereof.

### TECHNICAL FIELD

In the invention, it is crucial that bioactive substances including the pressed mass of berries or fruit will be gotten water-soluble form for pharmaceutical, herbal, nutrition and cosmetics industries as a liquid, powder, tablets, capsules, gel, cream or smoothie or as a part of them.

### BACKGROUND OF THE INVENTION

It is well known that berries and fruits are healthy. Berries and fruits are pressed for the juices, such as grape juice from grapes, but many of the useful substances are staying in the skin/peel of berry or fruit or other part of them or they are in a such form that the body cannot use them effectively. It is known that many skin/peel of berries and fruit peel include important bioactive substances, and the according to the invention they will get soluble form.

The invention can be used only in a particular part of the fruit or berry or the whole, for example, mixed with a blender. There is a big need for this invention, and it is very inventive that for using berries and fruits side flows this is not presented before.

A Report for utilizing possibilities of berries and fruits by-products flow in Finland, Katariina Roininen; Mirj a Mokkila, Sitra

http://www.sitra.fi/julkaisut/muut/VTTn%20marjaselvity %20.pdf retrieved 12 Mar. 2015) are shown in berries phenols extracts possibilities.

In the presentation Extraction, Isolation and Processing Technology on Berries. Zhang Bolin.is presented well berries extraction methods (http://www.helsinki.fi/ruralia/NWFPs-conference2013/pdf/Zhang%20Bolin.pdf retrieved 12 Mar. 2015). Hot water, ethanol and the enzyme-based methods are the most commonly used extraction methods.

In the patent publication CN103880806 Method for extracting from blueberry anthocyanin, Yi Caiyong; Zhao Wei presents the using of ethanol and petroleum ether in the extraction of blueberry.

In the publication An Optimized Method for Analysis of Phenolic Compounds in Buds, Leaves and Fruits of Black Currant (Ribes nigrum L.), Michael Vagiri; Anders Ekholm; Staffan Andersson, C.; Eva Johansson; Kimmo Rumpunen J. Agric. Food Chem. 2012, 60, 10501-10510 http://www.northsearegion.eu/files/repository/20140106103940_2013-SE-Enclosure10.pdf retrieved 12 Mar. 2015) presents the use of acetonitrile, formic acid and water and methanol, and ethanol and phosphoric acid, as a solvent for extraction in blackcurrants.

In the patent publication WO 2013123618 Process for the manufacture of the extract containing zeaxanthin and/or its esters, He, Qing; Zhao; Haining; Hugentobler, Max is shown in the manufacture of goji juice using of leaving goji berry waste with a solvent, ethyl acetate, hexane, heptane, petroleum ether, acetone, butane, methyl ethyl ketone, methanol, isopropyl alcohol or their mixture.

In the publication Comparisons of various extraction techniques for the determination of chemical constituents of Lycium barbarum fruit (Goji), Jirapat Namkaew; Ho-Hsien Chen; Sirikhae Pongswat; Songphon Chamdit (http://www.coop.rmutt.ac.th/?wpfb_d1=529 retrieved 12 Mar. 2015) presents goji berry extraction in 200 ml ethyl ether 55° C. for 8 hours, and 200 ml n-hexane at 85° C. for 8 hours.

### SUMMARY OF THE INVENTION

The present invention relates to a method of extraction of berry or fruit material as a drug, herbal, dietary supplement or cosmetic product ingredient, so that the berry or fruit material in the extraction solvent, typically by addition of hot water, in addition to the alkaline magnesium compound is used. The invention is an important berry and fruit side flows for getting bioactive substances water-soluble form, together with the alkaline magnesium compound.

### DETAILED DESCRIPTION OF PREFERRED EMBODIMENTS

Berry or fruit material (1a)) is from one or some of the following species, such as the grape (Vitis species), plum (Prunus domestica), wild blueberry and blueberry (Vaccinium species), lingonberry (Vaccinium vitis-idaea), cranberry (Vaccinium oxycoccos), crowberry (Empetrum nigrum), bog bilberry (Vaccinium uliginosum), cloudberry (Rubus chamaemorus), sea buckthorn (Hippophae rhamnoides), rowan (Sorbus), aronia (Aronia), black currant (Ribes nigrum), red currant (Ribes rubrum), white currant (Ribes rubrum rubrum species), gooseberry (Ribes uva-crispa), raspberry (Rubus idaeus), rose hip (Rosa canina), strawberry (Fragaria), goji (Lycium barbarum), acai (Acai), peach (Prunus persica), cherry (Cerasus), plum (Prunus), kiwi (Actinidia delicios), cucumber (Cucumis sativus), tomato (Solanum lycopersicum), apple (Malus), baobab (Adansonia), carob (Ceratonia siliqua), cape gooseberry (Physalis peruviana), mulberry (Morus alba) or/and camu camu (Myrciaria dubia).

In the markets there are many dried berrries and fruits, such as grapes or raisins, cranberries, apples, figs, dates, acai, cam camua, gojia, cherry, Inca berry, cranberry, mulberry, Schizandra berry powder, raspberries, strawberries, sea buckthorn , blueberries, lingonberries, black currants, red currants, green currants, white currants, tomatoes, apples, pears and rose hips.

There are many health benefits with magnesium. In the inventions it is used alkaline magnesium compound (FIG. 1b)), such as magnesium oxide, magnesium oxide and/or magnesium carbonate, typically as an aqueous solution (1 c)).

An extraction is one of the oldest and most important chemical process, such as production of tea beverages with hot water extraction. Now, the present invention is used to heat for extracting substances. For example, while the content of vitamin C can decrease in heat extraction, it is not a problem. Cloudberry (Rubus chamaemorus) contains vitamin C, which concentration is 3-4 times higher than oranges. By synthesis of vitamin C molecule, the price is a fraction compared to the cloudberry containing vitamin C. Vitamin C molecule is very advantageous to prepare on an industrial scale synthesis, and Vitamin C are added to many products. So the price is a very important thing, if only, for example by eating cloudberries to get the necessary amount of vitamin C, it would be very expensive. Another possibility is to even though the temperature must be kept high, the first to press the juice and then make extraction fruit according to the invention, and then extraction adding to the pressed juice.

Berries, fruits and vegetables contain many ingredients which are so complex and many times so that they can not be synthesized in practice. The key thing is to get these substances to the bioavailable form. Many people chew peel and seeds, and it can help in some amount, but in many cases but the materials are not soluble enough in water, such as when making tea it is needed to use hot water in the extraction.

Decanting is also a common chemical method. If necessary, the filtering can be made if needed.

FIG. 1 shows a method chart of the invention, at which point 1 a) fresh or dried berry or fruit plant material is contained in a soluble form, which can be a solution or part of solution was used to make powders, ointments, gels, lotions, tablets, capsules or smoothie, or part of it. Water is a solvent containing the alkaline magnesium compound and heated, allowed to cool, the FIG. 2 a) is dried grapes or raisins according to Example 2. In left of the FIG. 2a) it is used as the alkaline magnesium compound as a en extraction, and the dried grapes bioactive substances are gotten in water-soluble form.

### BRIEF DESCRIPTION OF THE FIGURES

FIG. 1

Dried or fresh berries or fruit material is FIG. 1 a) mixing with water 1 c), which contains an alkaline magnesium compound 1 b), and boiling in this mixture, the resulting mixture 1d) is allowed to cool and a berry and fruit materials will be gotten soluble form 1 e), which will be decanted or used wholly.

FIG. 2

FIG. 2 a) is made according the invention, in order to getting dried grapes (vine, Vitis), or raisins into soluble form and in 2 b) is made in the same way for the same a berry with hot water, but without the alkaline magnesium compound.

### EXAMPLES

### Example 1

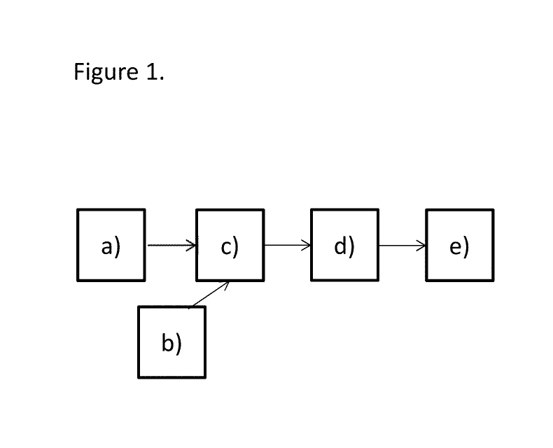

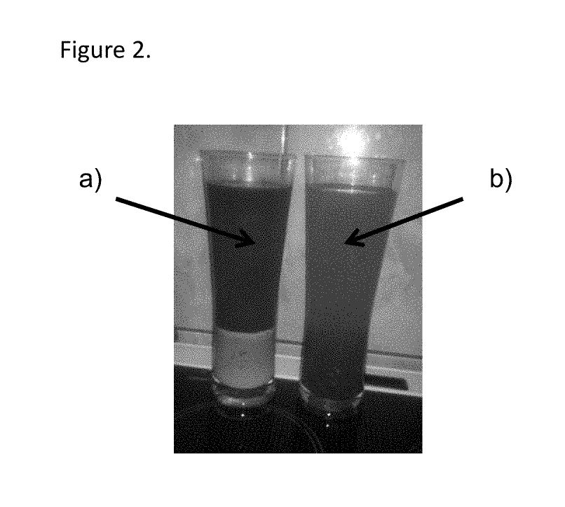

Dried grape, obtained from vine (Vitis), or raisins (FIG. 1 a) 50 g stirred with a blender in 1000 ml of water and 20 g of magnesium oxide. The boiling time was one hour, 100° C. and normal air pressure. The resulting mixture was allowed to cool, and the clear solution was separated the solution (FIG. 2 a)) was decanted and used as a health drink.

### Example 2

The dried sea buckthorn (Hippophae rhamnoides) (FIG. 1a) 10 g was stirred with 1 liter of boiling water containing 10 g of magnesium oxide. The boiling time was hour period, 100° C. and normal air pressure, for 30 minutes, the mixture was stirred with a blender for 20 seconds. The resulting mixture was allowed to cool, and the clear solution was separated the solution (FIG. 2 a)) was decanted and used as a health drink.

Those skilled in the art it is clear that the invention is not limited to embodiments in detail above, but the use of other berries or fruits. Those skilled in the art it is clear that different extraction temperatures and times, as well as solvents, solvent mixtures, and pressures within the scope of the present invention, such as obtaining a powder from the liquid with known methods, such as spray and freeze-drying techniques.

According changing rules and a new PCT rule 1 Jul. 2010 46.5, 66.8, 72.2 (c-bis) there is presented paragraphs where the changes are in this priority application FI20150078.

In the intermediate decision of this priority application FI20150078 by National Board of Patents and Registration of Finland, it was presented the next reference publications.

The patent publication CN 101298449, Zhao Quancheng, Novel preparation of myricetin, The invention is a method for industrially preparing myricetin from stems and leaves of Ampelopsis sinica (Miq.) W. T. Wang of vitaceae Ampelopsis Michx or other plants of the vitaceae Ampelopsis.

The patent publication KR 20020086117, Kim Eun Ju, Kim Ui Su, Park Myeong Gyu, Yoo Tae Hui, Antioxidant containing alkali extract from mixture of defatted rice bran and onion skin.

The patent publication US 20070166407, Akiko Tanaka, William Bradley, Plant extracts and uses thereof. Plant material is selected from the group consisting of leaves, needles, bark, wood, stalks, sheath and cones.

The patent publication US 20100111994, Francis Tufaro, Akiko Tanaka, Michael P. Burrows, William G. Bradley, Joanna Peterkin, Anti-bacterial composition and method of using same. The invention is an alkaline extract of pine cones with anti-bacterial activity.

There are market researches. Such a Report for utilizing possibilities of berries and fruits by-products flow in Finland, https://www.sitra.fi/julkaisut/muut/VTTn%20marjaselvitys%20b. pdf Selvitys marjojen ja marja sivuvirtojen hyödyntamispotentiaalista Suomessa, This report was made by VTT, VTT is the Technical Research Centre of Finland, and there are big possibilities with the by-products of berries.

There are many chemistry researches for using the pressed waste, by-products of berries and fruits. Such the Report of phenolic compounds isolation technologies from berries, http://www.sitra.fi/julkaisut/muut/marjojen%20fenolisten.pdf Selvitys marjojen fenolisten yhdisteiden eristämisen teknologiasta. This report was made by VTT and Lappeenranta University of Technology.

The method according to the invention was not presented before in any fruit or fruit extraction, even though these have very big needs. The raw material availability is very good, and for a long time it been studied using of these by-products, pomace, which including important substances. There are big, long well known needs for using pressed mass, by-products of berries and fruits in valuable products.So it is inventive that there have been long and well known need for this very abundant and cheap material for using, but this is not presented in berries and fruits, which are big health polyphenolic sources. Current technologies are using hot water, methanol or/and ethanol. Hot water is not enough efficient, methanol is toxic and ethanol is too polar, and also the target is also so called Green chemistry, where is prefer to use aqueous solution than organic solvents. This invention is so new and inventive.

1. A method of extraction berries including substances (1a)) using of heat in the pharmaceutical, dietary supplement, herbal, or cosmetic product ingredients, is characterized in that in the extraction of the substances (1 a)) use the solvent (1 c)), typically water, and also an alkaline magnesium compound (1b)).

2. The method according to the claim 1 is characterized in that the magnesium containing alkaline compound (1b)) is magnesium oxide, magnesium hydroxide, magnesium carbonate, dolomite, brucite, talc or magnesite, or mixtures thereof.

3. The method according to the claim 1 is characterized in that the magnesium-containing compound (1b)) at the beginning is magnesium, which reacts with the solvent (1 c)), typically water, to form the alkaline magnesium compound.

4. The method according to the claim 1 is characterized in solvent (1 c)) is water, ethanol, ethanol aqueous solution or an oil such as vegetable oil.

5. The method according to the claim 1 is characterized in that the berry or fruit (1 a) contained in is the whole and/or pressed, dried or fresh in different milling sizes.

6. The method according to the claim 1 is characterized in that the berry and fruit (1 a)) is derived from one or some of the following species, such as grape (Vitis species), plum (Prunus domestica) wild blueberry and blueberry (Vaccinium species), lingonberry (Vaccinium vitis-idaea), cranberry (Vaccinium oxycoccos), crowberry (Empetrum nigrum), bog bilberry (Vaccinium uliginosum), cloudberry (Rubus chamaemorus), sea buckthorn (Hippophae rhamnoides), rowan (Sorbus), aronia (Aronia), black currant (Ribes nigrum), red currant (Ribes rubrum), white currant (Ribes rubrum rubrum species), gooseberry (Ribes uva-crispa), raspberry (Rubus idaeus), rose hip (Rosa canina), strawberry (Fragaria), goji (Lycium barbarum), acai (acai), peach (Prunus persica), cherry (Cerasus), plum (Prunus), kiwi (Actinidia delicios), cucumber (Cucumis sativus), tomato (Solanum lycopersicum), apple (Malus), baobab (Adansonia), carob (Ceratonia siliqua), cape Gooseberry (Physalis peruviana), mulberry (Morus alba) and camu camu (Myrciaria dubia).

7. The method according to the claim 1 is characterized in berries or fruit (1 a)) is at first compressed and them which are destroyed in the hot temperature, such as vitamin C is used separately and/or added to the extract (1 e)).

8. The method according to the claim 1 is characterized by a mixture of (d 1)) used in the extraction temperature is 0-100° C. and also higher than 100° C. change in changing pressure, typically 100° C. at atmospheric pressure.

9. The method according to the claim 1 is characterized by the extraction mixture (1 d)), (d) time 1-600 min, typically 20 min.

10. The method according to the claim 1 is characterized by the extraction of the extract 1 e)) is filtered and/or is decanted and is used in solution, powder, tablet, capsule, gel, lotion, ointment, smoothie or a part of it.
